(19) United States
(12) Patent Application Publication
(10) Pub. No.: US 2025/0205478 A1
(43) Pub. Date: Jun. 26, 2025

(54) ELECTRODE ASSEMBLIES HAVING EMBOSSED NERVE CONTACT ELEMENTS AND ASSOCIATED METHODS

(71) Applicant: The Alfred E. Mann Foundation for Scientific Research, Valencia, CA (US)

(72) Inventors: Christopher Reed Jenney, Valencia, CA (US); William Andrew Brandt, Castaic, CA (US)

(73) Assignee: The Alfred E. Mann Foundation for Scientific Research, Valencia, CA (US)

(21) Appl. No.: 18/985,448

(22) Filed: Dec. 18, 2024

Related U.S. Application Data
(60) Provisional application No. 63/613,585, filed on Dec. 21, 2023.

(51) Int. Cl. A61N 1/05

(52) U.S. Cl. CPC A61N 1/0556

(57) ABSTRACT

An electrode contact having an embossed surface for use in a stimulation electrode and methods of making the embossed electrode contact and stimulation electrode.

### CROSS-REFERENCE TO RELATED APPLICATION

This application claims the benefit of U.S. Provisional Application No. 63/613,585, filed Dec. 21, 2023, and entitled “Electrode Assemblies Having Embossed Nerve Contact Elements and Associated Methods,” which is incorporated herein by reference.

### FIELD OF THE INVENTION

The present invention relates generally to nerve or tissue stimulation apparatus and methods of making the same and, in particular, the present invention provides methods for manufacturing electrode assemblies having nerve contact elements that are embossed to improve mechanical and electrochemical performance.

### BACKGROUND

Various types of devices have been developed for implantation into the human body to provide various types of health-related therapies and/or monitoring. Examples of such devices, generally known as implantable medical devices (“IMDs”), include cardiac pacemakers, cardioverter/defibrillators, cardiomyostimulators, various physiological stimulators including nerve, muscle, and deep brain stimulators, various types of physiological monitors, and drug delivery systems, just to name a few. For the purposes of this application, reference will be made only to implantable neurostimulators, such as implantable pulse generators (“IPGs”), it being understood that the principles described herein may have applicability to other implantable medical devices as well.

IPGs are often used in the context of neuromodulation therapy, and, in general, comprise a hermetically sealed housing that houses stimulation circuitry, and a header which is mounted on the IPG housing. The header includes a connector, and the connector is electrically coupled to electronics within the housing by way of feedthrough pins which extend from the header connector and into the housing. The connector defines a receptacle configured for receipt of a proximal end of a stimulation lead, and an electrode disposed at a distal end of the stimulation lead provides an interface for transmitting electrical signals between the stimulation circuitry and an anatomical target (e.g., a nerve).

Stimulation electrodes generally comprise a biocompatible, pliable, electrically insulative substrate and at least one electrode contact (“contacts”). The contacts are formed from an electrically conductive material, such as, e.g. platinum-iridium, are electrically coupled to the IPG stimulation circuitry via the lead, and may be partially embedded in (or otherwise attached to) the substrate so that at least a portion of the contact can interface with the anatomical target.

In practice, stimulation electrodes come in a variety of configurations to accommodate different applications. For example, a non-exhaustive list of stimulation electrodes known in the prior art includes cuff electrodes (i.e. nerve cuffs) as disclosed in U.S. Pat. No. 9,283,394; paddle electrodes (i.e. electrode arrays) as disclosed in U.S. Pat. Pub. 20120209285A1; linear electrodes (e.g. percutaneous electrodes) as disclosed in U.S. Pat. No. 8,650,747; and helical electrodes as disclosed in U.S. Pat. No. 4,573,481.

One of the most common implantable electrodes to stimulate the peripheral nervous system is the nerve cuff. Nerve cuffs are configured to gently wrap around the epineural surface of a nerve trunk, and two common applications for the nerve cuff include stimulation of the hypoglossal nerve to treat obstructive sleep apnea and stimulation of the vagus nerve to treat epilepsy. Some nerve cuffs are pre-shaped to a furled state, are movable to a slightly unfurled state, and may be unfurled into a flat state. The electrode contacts of nerve cuffs typically comprise flat, rectangular bodies of conductive material, although other contact geometries such as hat-shaped, ring-shaped, or coil-shaped contacts, are also possible. In any case, the maximum size of an electrode contact is limited by the surface area available on an interior face of the cuff, and the maximum current density which can be transmitted through a contact is limited by the effective surface area of the contact.

Like nerve cuffs, helical electrodes are also used for peripheral nerve stimulation and have similar applications to traditional nerve cuffs. Helical electrodes comprise a coil-shaped substrate which is configured to wrap around the target nerve, and contacts of helical electrodes typically are spaced along an interior surface substrate to deliver electrical stimulation to the nerve at discrete locations along its length. In one aspect, helical electrodes and nerve cuffs can be “self-sizing” in that some can flexibly accommodate a range of nerve diameters, however, with respect to charge delivering capacity, the contacts of helical electrodes face the same limitations as nerve cuffs.

Linear electrodes and paddle electrodes can be utilized for stimulating the nerves in the spinal cord. Both are frequently used for stimulation of the sacral nerves or spinal cord, but they differ in their construction. Linear electrodes generally comprise an elongate, cylindrical, and flexible electrode body disposed at the distal end of a lead, and one or more ring-shaped contacts are spaced apart along the body and separated by insulative material. Paddle electrodes, on the other hand, generally comprise a flat, insulative substrate having an array of contacts (sometime referred to as “electrode arrays”) disposed on a front face of the substrate. Accordingly, the low-profile construction of linear electrodes can be advantageous for minimally invasive applications, whereas paddle electrodes can provide for greater selectivity and specificity of stimulation due to the distribution of the electrode contacts while requiring more invasive surgical implantation.

Processes for manufacturing implantable medical devices (IMDs) continually stand to be improved for the purposes of minimizing product size, lowering production costs, increasing product longevity, and improving product performance, among others. There is also a general desire to minimize the size of the IMD components without compromising the efficacy and efficiency of deliverable treatment. This is also true for IMDs such as stimulation leads, electrodes, and, in particular, contacts for stimulation electrodes.

### SUMMARY

The present inventors have determined that nerve stimulation electrodes (e.g., nerve cuffs, helical electrodes, linear electrodes, and paddle electrodes, etc.), are susceptible to improvement. For example, the present inventors have determined that certain electrically conductive materials with otherwise desirable properties (e.g., platinum-iridium) do not bond well with non-conductive substrate materials that have desirable mechanical properties (e.g., silicone). A less-than-optimal bond, coupled with stresses that occur when the electrode body is manipulated (e.g., furled, unfurled, or twisted), may lead to delamination of the contact, exposure of internal components not intended for direct bodily fluid contact, and, in some instances, dislodgement of the contact from the electrode body. Exposure of the internal components to bodily fluid may result in electrochemically driven oxidation of the cables during stimulation pulses and an increased risk of galvanic corrosion between the contacts and cable materials in the crimp joints connecting the cables to the contacts. In addition, exposure of the normally completely-covered rear side of the contacts to bodily fluid reduces the effective charge density which can result in less effective stimulation. Accordingly, the present inventors have determined that it would be desirable to provide stimulation electrodes that, among other things, include contacts configured to reduce the likelihood of delamination.

Other issues identified by the present inventors are associated with the effective surface area of electrode contacts (i.e., the surface area value accounts for the surface roughness of the contacts as well as the geometric boundaries defined an outer periphery of the contacts). The miniaturization of the electrode contacts enhances the spatial resolution and selectivity of stimulation. On the other hand, the smaller the surface area of the contact, the lower the charge transfer and the higher the impedance of the phase boundary. To overcome this contradiction, the present inventors have determined that it would be desirable to increase the effective surface area of electrode contacts (without increasing their geometric size) to increase the amount of stimulation energy that can be safely delivered, reduce electrode-electrolyte impedance, and reduce power consumption by the stimulation circuitry.

Historically speaking, known methods for enhancing the effective surface area of electrode contacts have involved the creation of macroporous structures on a surface of the contact through sintering, etching, electrochemical plating, and plasma-enhanced chemical or physical vapor deposition. However, such methods possess deficiencies that stand to be improved. These methods can be difficult to perform at scale, require complex fabrication and expensive equipment, and, in instances where the contacts comprise an irregular geometry (such as cylindrical rings, helical coils, or otherwise), the implementation of these methods can become increasingly cumbersome.

Thus, the present invention seeks to overcome deficiencies of the prior art by providing an inexpensive and robust method for manufacturing electrode leads, and, in particular, contacts having a textured surface that increases mechanical and electrochemical performance. Further the present invention seeks to provide a method that is both scalable and widely applicable electrodes of varying constructions.

In one aspect, the present invention provides a method for embossing one or more electrode contacts (“contacts”) with a surface pattern. The contacts are formed from an electrically conductive material (or “workpiece”), and the surface pattern is configured to either increase the effective surface area of the contact(s), increase the charge-delivering capacity, lower electrode-electrolyte impedance, and/or improve bonding strength between the contact(s) and a substrate. After an embossing process, the contacts are assembled with a biologically compatible, elastic, and electrically insulative substrate to form an electrode, and a stimulation lead may electrically couple the electrode to stimulation circuitry housed within an IPG.

In another aspect, the present invention provides an electrode lead configured for delivering electrical stimulation to a nerve. The electrode lead comprises an elongate lead body having proximal end and a distal end, and an electrode is affixed to the distal end. The electrode comprises a biologically compatible, elastic, electrically insulative substrate, and a plurality of contacts are secured to a front face of the substrate. The contacts are formed from electrically conductive material, each contact including a rear outer surface in connection with the substrate, and a front outer surface for interfacing with the nerve, wherein at least one of the front or rear outer surfaces of the contact is embossed with a surface pattern configured to increase the effective surface area, increase the charge-delivering capacity, lower electrode-electrolyte impedance, and/or improve bonding strength between the contact and the substrate.

In instances where the contacts are formed from cylindrical rings (or some other geometry) of electrically conductive material, the present invention provides a method for embossing at least a portion of an outer surface of the contacts with a surface pattern. The method comprises rolling the cylindrical rings of electrically conductive material across a textured tooling surface, and this method may herein be referred to as “roll-embossing”.

In some embodiments, the method for roll-embossing may include the formation of a subassembly between the cylindrical rings of electrically conductive material and a support pin. The support pin is inserted through the rings to prevent the rings from collapsing during the roll-embossing process, and the pin may also drive rotation of the ring upon the textured tooling surface.

For applications in which ring-shaped contacts are desirable, such as, e.g., when forming a linear electrode, the method comprises assembling the embossed cylindrical rings with a biologically compatible, electrically insulative substrate to form the electrode.

In other embodiments when substantially flat contacts are desired, such as, e.g., when forming a nerve cuff, a paddle electrode, a helical electrode, or otherwise, the method may further comprise the optional step of flattening the embossed cylindrical rings into substantially rectangular embossed contacts. The substantially rectangular embossed contacts are then assembled with the substrate to form the electrode, the electrode may be connected to a distal end of a lead, and a proximal end of the lead may electrically couple the electrode to stimulation circuitry housed within an IPG.

In another embodiment, the present invention provides an electrode lead. The electrode lead includes an elongate lead body having a proximal end and a distal end, and a biologically compatible, elastic, electrically insulative substrate is affixed to the distal end. The substrate includes a front surface having a plurality of windows and a plurality of contacts are assembled with the substrate such that they are disposed within the windows. Each contact includes a front surface for interfacing with a nerve, and the front surfaces are embossed with a surface pattern. The respective front surfaces of the embossed contacts each define an effective surface area that is bounded by the outer perimeter (“ESA”) and that is at least twice the smooth surface area (“SSA”) that is defined by the outer perimeter the embossed surface, i.e., the area of a perfectly smooth surface having the size and shape of the embossed surface. The present invention also includes systems with an implantable pulse generator (or other implantable stimulation device) in combination with such an electrode lead.

Systems and methods in accordance with the present invention are shown and described herein in the context of processes for manufacturing electrode contacts (“contacts”) having flat, ring-shaped, or helical contact geometries, it being understood that implementations of present invention could be similarly applied to other configurations not explicitly described herein. Similarly, contacts manufactured in accordance with methods of the present invention may be incorporated into various types of stimulation electrodes with exemplary applications shown and described herein including nerve cuffs, helical electrodes, linear electrodes, and paddle electrodes, it being understood that implementations of present invention could be similarly applied to other electrode configurations not explicitly described herein.

### BRIEF DESCRIPTION OF THE DRAWINGS

Detailed descriptions of exemplary embodiments will be made with reference to the accompanying drawings.

FIG. 1 is a plan view of a stimulation system including a stimulation lead with a nerve cuff electrode in accordance with one embodiment of a present invention.

FIG. 2 is an isometric view of the stimulation lead illustrated in FIG. 1.

FIG. 3 is a cut-away anatomical drawing of the head and neck area illustrating the muscles that control movement of the tongue, the hypoglossal nerve (HGN) and its branches that innervate these muscles, and the nerve cuff electrode illustrated in FIG. 1 on the HGN trunk.

FIG. 4 is a view of the nerve cuff electrode illustrated in FIG. 1, in an unfurled and flat state, having spaced apart electrode contacts.

FIG. 5 is a cross-sectional view of nerve cuff electrode illustrated in FIG. 1.

FIG. 6 is a side view of a stimulation lead with a helical electrode in accordance with one embodiment of a present invention wrapped around a nerve.

FIG. 7 is an enlarged view of a portion of the helical electrode illustrated in FIG. 6 in an unfurled and flat state.

FIG. 8 is a partial cross-sectional view of a spinal cord and a stimulation lead with a linear electrode in accordance with one embodiment of a present invention connected to an implantable pulse generator (IPG) used for spinal cord stimulation.

FIG. 9 is an isolated view of the linear electrode stimulation lead and IPG illustrated in FIG. 8.

FIG. 10 is an enlarged view of the linear electrode illustrated in FIG. 8.

FIG. 11 is a view of a stimulation system with a paddle electrode in accordance with one embodiment of a present invention connected at its proximal end to an IPG.

FIG. 12 is an isometric view of a tubular workpiece of conductive material in a pre-processed state and used to make an electrode contact.

FIG. 13 is an end view of the tubular workpiece of FIG. 12.

FIG. 14 is a top view of a tooling surface prior to undergoing a surface treatment.

FIG. 15 is a side view of a tooling surface prior to undergoing a surface treatment.

FIG. 16 is a side view of a tooling surface undergoing an exemplary surface treatment.

FIG. 17 is a series of views depicting a tooling surface undergoing a surface treatment, an embossing/flattening process for a tubular workpiece in accordance with one embodiment of a present invention, and magnified images of the surfaces of the tooling surface and the tubular workpiece before and after the surface treatment and embossing process, respectively.

FIG. 18 is a side view of a substantially flat workpiece of conductive material undergoing a stamping embossing process in accordance with one embodiment of a present invention.

FIG. 19 is a side view of a tubular workpiece of conductive material undergoing a rolling embossing process in accordance with one embodiment of a present invention.

FIG. 20 is a side view of another tubular workpiece of conductive material undergoing an embossing process in accordance with one embodiment of a present invention.

FIG. 21 is a side view of the tubular workpiece illustrated in FIG. 20 after having been embossed and flattened into a substantially flat contact.

FIGS. 22A and 22B are views depicting the embossing of a tubular workpiece which is supported by a pin in accordance with one embodiment of a present invention.

FIGS. 23A and 23B are views depicting the embossing of a tubular workpiece which is supported by a pin in accordance with one embodiment of a present invention.

FIG. 24 is a top view of an embossed surface pattern in accordance with one embodiment of a present invention.

FIG. 25 is a top view of a surface pattern in accordance with one embodiment of a present invention.

FIG. 26 is a top view of a surface pattern in accordance with one embodiment of a present invention.

FIG. 27 is a top view of a surface pattern in accordance with one embodiment of a present invention.

FIG. 28 is an enlarged, isometric view of a portion of the surface pattern shown in FIG. 24.

### DETAILED DESCRIPTION

The following is a detailed description of the best presently known modes of carrying out the inventions. This description is not to be taken in a limiting sense but is made solely for the purpose of illustrating the general principles of the inventions.

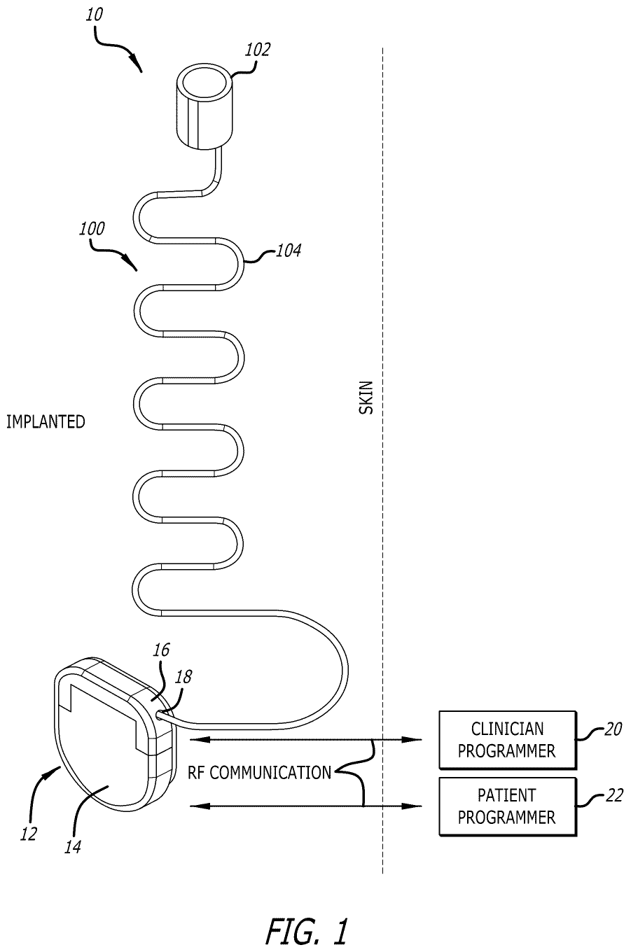

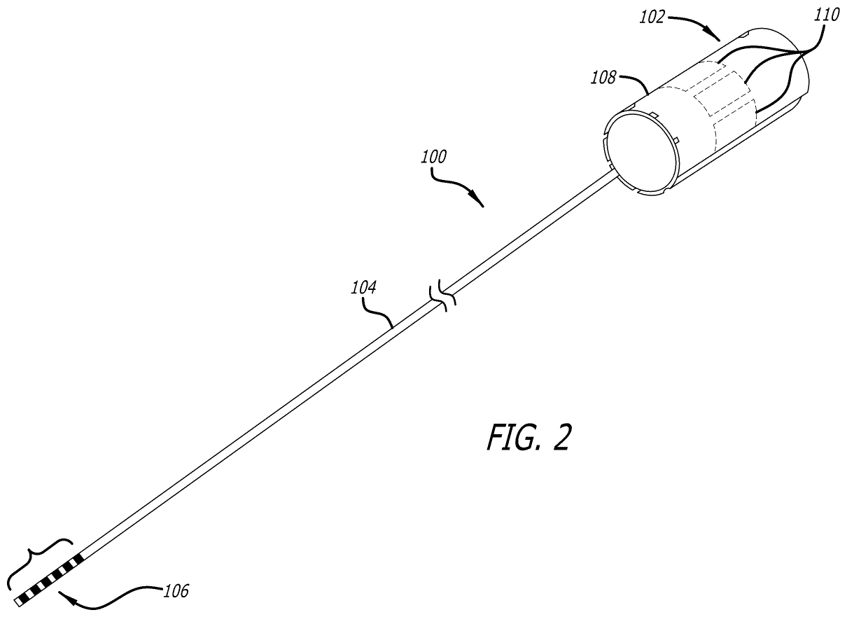

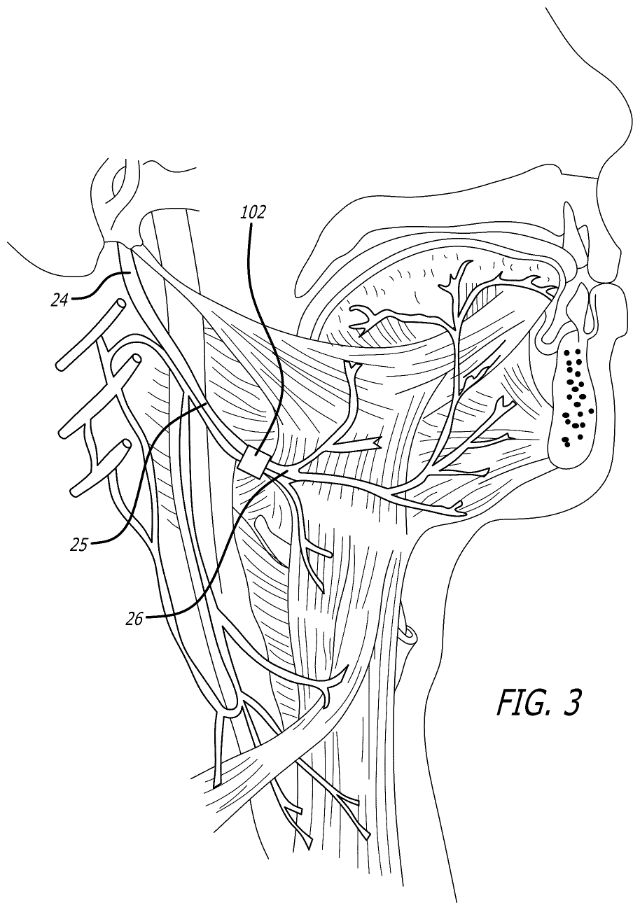

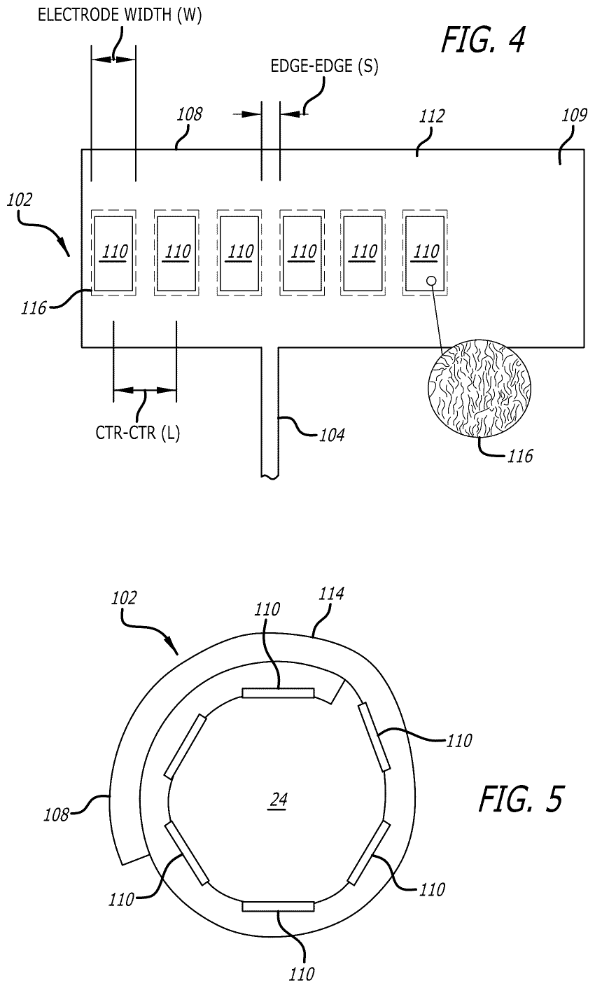

Referring to FIGS. 1 and 2, a stimulation system 10 in accordance with one embodiment of a present invention includes an electrode lead 100 and an implantable stimulator such as the implantable pulse generator (“IPG”) 12. A clinician's programming unit 20, a patient programmer or patient remote 22 and/or an IPG charger (not shown) may also be provided in some instances. Suitable IPGs, clinician's programming units and patient remotes are described, for example, in U.S. Pat. Pub. No. 2022/0313987A1. The electrode lead 100 of FIG. 1 includes a nerve cuff electrode 102 (also referred to as “nerve cuff” or “cuff electrode” in this disclosure), and a lead body 104 couples the nerve cuff electrode 102 to the IPG housing 14 by way of lead connector 106 (FIG. 2) on the proximal end of the lead body 104. The lead connector 106 is configured to mate with a connector receptacle 18 on a header 16 of the IPG 12. FIG. 2 shows the nerve cuff electrode 102 and the entirety of electrode lead 100. The nerve cuff electrode 102 is comprised of a pliable, electrically insulative cuff substrate 108, and one or more electrode contacts 110 are attached within the inside surface of the cuff substrate 108. The one or more contacts 110, which may be spaced apart, are used to electrically stimulate a nerve that is encircled by the nerve cuff electrode 102. The exemplary substrate 108 (and nerve cuff electrode 102) is pre-set (or “pre-shaped”) to the furled (or “curled”) state illustrated in FIGS. 1 and 2, is movable to a slightly unfurled state, and may be unfurled into the flat state illustrated in FIG. 4 (discussed below).

Turning to FIG. 3, nerve cuff electrode 102 is configured in such a manner that it may be circumferentially disposed around a nerve 24, such as the hypoglossal nerve (HGN), at either the HGN trunk 25 or a HGN branch 26 (e.g., the HGN GM branch). The lead body 104 may include one or more S-shaped sections (as shown in FIG. 1) to provide strain relief or may be straight. The S-shaped sections accommodate body movement at the location within the neck where the lead body 104 is implanted, thereby reducing the likelihood that the target nerve (in this case, the HGN) will be damaged due to unavoidable pulling of the electrode lead 100 that may result from neck movements. The accommodation provided by the S-shaped sections also reduces the likelihood of fatigue damage to the lead. Additionally, although the exemplary stimulation system 10 includes a singular electrode lead 100, other embodiments may include a pair of electrode leads 100 for bilateral nerve stimulation and an IPG with two connector receptacles as shown in FIG. 11.

FIG. 4 shows a top view of the inside surface of the nerve cuff electrode 102 in a flat, unfurled state and attached to the lead body 104. The embodiment of the nerve cuff 102 shows six rectangular-shaped electrode contacts 110 associated with the inner surface 112 of the substrate 108. The six electrode contacts 110 can be collectively called an “electrode array”. Each of the contacts 110 has an embossed front surface 116 that is exposed on the inner surface 112 of the substrate 108. As used herein, an “embossed” surface is a three-dimensional surface with a raised and/or depressed texture that has been created by applying pressure to the surface. The embossed surface 116 may be created with one of the exemplary processes described below, or through the use of any other suitable process.

FIG. 5 shows a cross-sectional view of the nerve cuff electrode 102 of FIG. 4. The nerve cuff is wrapped around nerve 24, in a normal furled state. In this furled state, the nerve cuff has an inner surface 112 and outer surface 114. Normally, nerve cuff electrode 102 is made of substrate 108 that has some memory, i.e., that while the nerve cuff can be unfurled and made flat as shown in FIG. 4, due to memory, it will naturally return to its resting state of being furled as shown in FIG. 5. The embodiment of the nerve cuff shown in FIG. 5 has a distal flap 109 that extends from the last contact 110 and thereby can overlap the other end of the cuff edge. The exemplary contacts 110 have the aforemention embossed front surface 116, which is exposed, as well as a rear surface 117 attached to the substrate 108.

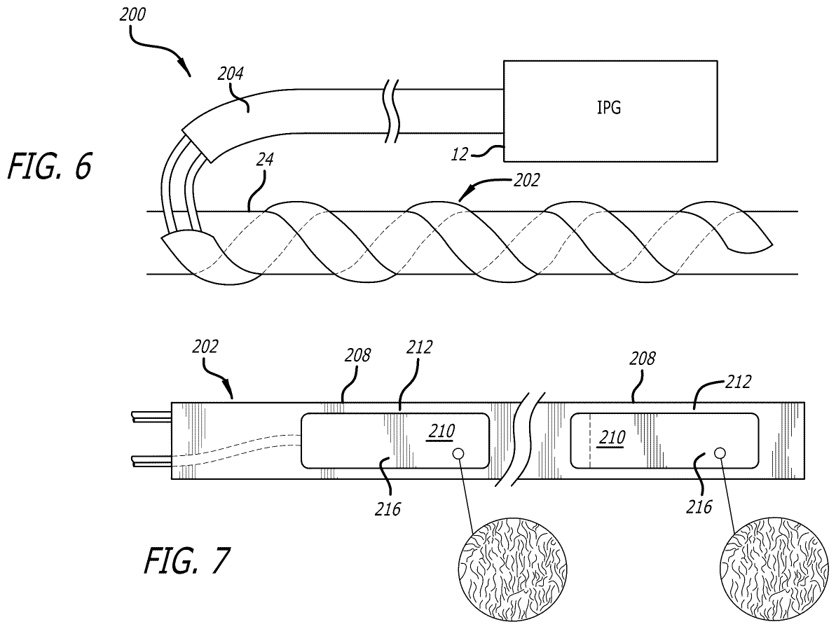

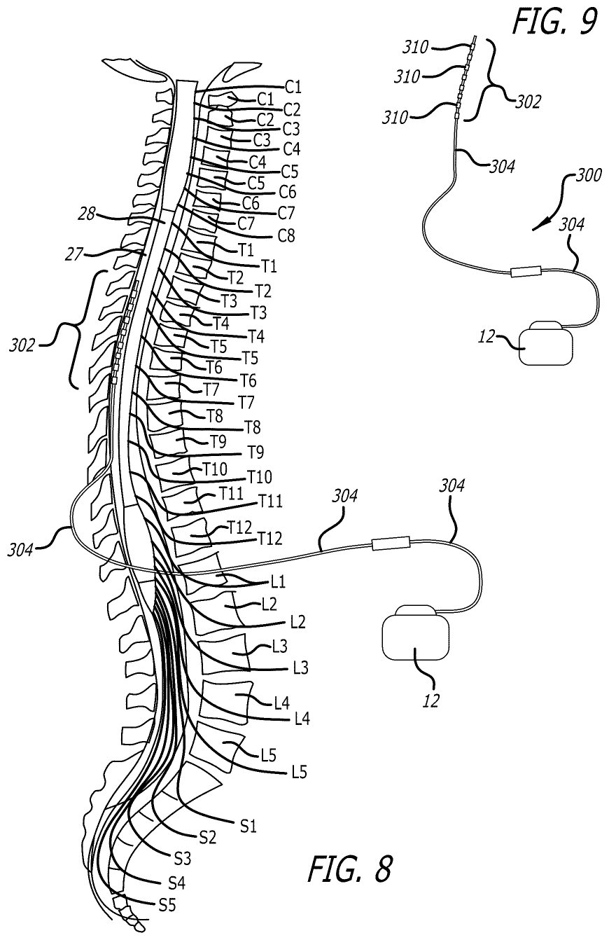

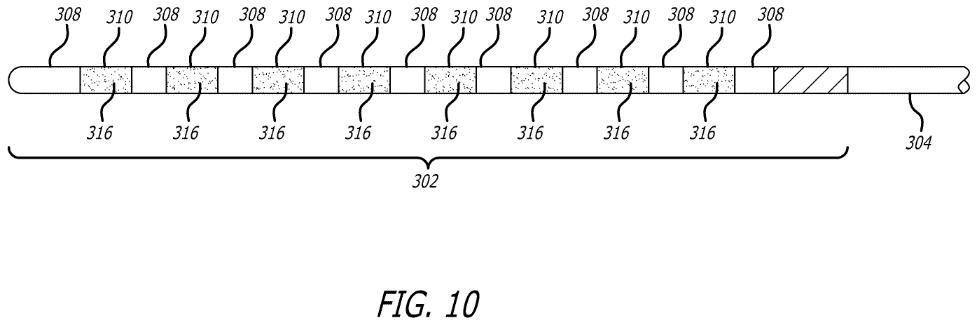

FIGS. 6-11 show a collection of other electrode configurations that include contacts with embossed surfaces, as is described in greater detail below. Specifically, FIGS. 6-7 show a stimulation lead 200 with a helical electrode 202, FIGS. 8-10 show a stimulation lead 300 with a linear electrode 302, and FIG. 11 shows a stimulation lead 400 with a paddle electrode 402 (also referred to as an “electrode array”).

With reference to FIGS. 6-7, helical electrode lead 200 includes a lead body 204 having a proximal end connected to a stimulator, such as an IPG 12, and a distal end of the lead body 204 is connected to the helical electrode (or “nerve cuff electrode”) 202. The helical electrode 202 includes a substrate 208 having a helical shape configured to spirally wrap around the nerve 24. The exemplary substrate 208 (and nerve cuff electrode 202) is pre-set (or “pre-shaped” to the furled (or “curled”) state illustrated in FIG. 6, is movable to a slightly unfurled state, and may be unfurled into the flat state illustrated in FIG. 7.

Referring to FIG. 7, the substrate 208 of helical electrode 202 has an interior face 212 that includes a plurality of rectangular electrode contacts 210. Contacts 210 are attached or partially embedded in the interior face 212 of substrate 208 such that embossed front surfaces 216 of contacts 210 protrude slightly outwardly from the interior face 212 of the substrate 208. The contacts 210 are used to transmit electrical stimulation current to and/or from the nerve 24. Because of memory in the substrate material, a flattened helical electrode will tend to return to its resting pre-set helical shape shown in FIG. 6.

Turning to FIGS. 8-10, linear electrode lead 300 is shown in the context of an exemplary application for spinal cord stimulation. The linear electrode lead 300 includes a lead body 304, which can be connected to an IPG 12 at a proximal end of the lead body 304, and the linear electrode 302 is disposed at a distal end of the lead body 304. Linear electrode 302 comprises a thin, elongate, and generally cylindrical substrate 308, and a plurality of ring-shaped contacts 310 are spaced apart along the distal part of the electrode lead 300. The ring-shaped contacts 310 have an embossed front surface 316 that extends completely around the cylindrical substrate 308. The plurality of ring-shaped contacts 310 also may be powered independently by the stimulation circuitry, thereby allowing stimulation to be supplied at discrete locations along the spinal cord 28. The relatively unobtrusive shape of the linear electrode can make it advantageous for minimally invasive applications, such as for insertion into the epidural space 27 proximate the spinal cord 28, and the cylindrical ring-shaped array of electrode contacts 310 allow for stimulation to be distributed along the distal portion of the linear electrode 302.

Referring to FIG. 11, paddle stimulation electrode lead 400 comprises a lead body 406 having a proximal end connected to the IPG 12, and a distal end connected to the paddle electrode 402. Paddle electrode 402 comprises a generally flat, substantially rectangular (i.e. “paddle-shaped”) substrate 408 formed from a biocompatible, electrically insulative material. A plurality of contacts 410 with respective embossed front surfaces 416, which are formed from electrically conductive material, may be partially embedded in- or otherwise adhered to a front face 412 of the substrate 408, the front face 412 being configured and adapted to interface with the target tissue (e.g., a nerve). Similar to linear electrode 302 discussed above, the paddle array of electrode contacts 410 is often used for central nervous system (CNS) stimulation, such as spinal cord stimulation, and delivery of stimulation through each contact 410 may be independently controlled by the stimulation circuitry to deliver programmable stimulation current or voltage amplitudes at each contact. Paddle electrodes often contain a plurality of contacts 410 arranged in one or more rows which allows for increased specificity of stimulation. In this embodiment, there are two rows of contacts 410, each row having eight contacts. The eight contacts in a row may sometimes be referred to as an “array” of electrode contacts.

In each of the aforementioned electrode configurations (i.e. nerve cuff electrode 102, helical electrode 202, linear electrode 302, and paddle electrode 402), there is a general desire to minimize the size of the electrically conductive contacts 110, 210, 310, 410 to enable packing of a greater number of contacts into a finite substrate surface space. Minimizing the size of the contacts can accommodate a smaller electrode body, thus allowing for a less invasive surgery, and smaller contacts are capable of delivering more precisely targeted stimulation. Increasing the number of contacts within a small space yields an unwanted result: electrical impedance through each contact will be high, since each contact will have a geometric surface area that will necessarily be small. One way to mitigate this result is to increase the effective surface area of the contact so that electrical impedance is reduced. More particularly, the greater the effective surface area of a contact, the more charge that may be delivered efficiently through the contact.

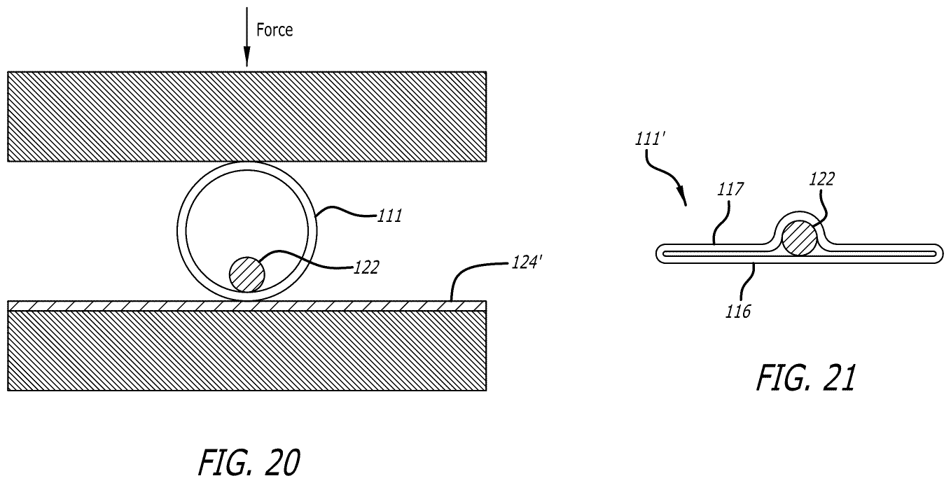

To achieve a greater effective surface area for the same apparent geometric contact surface size, the present invention provides a method for embossing the surfaces of electrode contacts 110, 210, 310, and 410 to create the embossed surfaces 116, 216, 316, 416 and thereby increase the effective surface areas of the electrode contacts (without increasing their apparent geometric size). The embossing method may be applied to ring-shaped bodies of conductive material to form embossed ring-shaped contacts, such as contacts 310 of linear electrode 302 in FIGS. 8-10, or the method may be applied to contacts of other geometries, such as flat contacts. In some embodiments, ring-shaped bodies of conductive material (or “tubular workpieces”) 111 (FIG. 20) may be embossed and subsequently flattened to form flat, generally rectangular embossed workpieces 111′ (FIG. 21). The embossed workpieces 111′ are then assembled with a biocompatible, pliable, insulative substrates 108, 208, 308, 408 to form electrodes 102, 202, 302, 402 (or electrode leads 100, 200, 300, 400), wherein the embossed workpieces 111′ form the contacts 110, 210, 310, or 410.

The substrates 108, 208, 308, 408 may be formed from any suitable biocompatible material and are electrically insulative and pliable. The substrates 108, 208, 308, 408 should be pliable enough to allow a clinician to implant, furl, bend and/or unfurl the electrodes 102, 202, 302, 402 for placement on the nerve 24 or spinal cord 28, and the substrate materials should also be resilient enough to cause the electrodes 102, 202, 302, 402 to return to their pre-shaped states when the force is removed. By way of example, but not limitation, suitable substrate materials include silicone, polyurethane, and styrene-isobutylone-styrene (SIBS) elastomers. Further, suitable materials for the contacts 110, 210, 310, 410 include, but are not limited to, platinum-iridium and palladium.

Referring back to FIGS. 2 and 4-5, the substrate 108 in the exemplary nerve cuff electrode 102 includes an inside surface 112 that faces the nerve 24 (e.g. the HGN trunk or branch) and an outer surface 114 that will face away from the nerve 24. The embossed contacts 110, which are formed from conductive material, are located on or embedded into the front layer 112 of substrate 108.

The contacts 110 each have the aforementioned exposed embossed front surface 116 and a rear surface 117 that is attached to or embedded into the substrate 108. Following an embossing process, which is described below, embossed front surfaces 116 of the contacts 110 have an effective surface area (“ESA”) that is at least twice the smooth surface area (“SSA”) that is defined by the outer perimeters the embossed front surfaces 116 of the associated contacts 110, i.e., the areas of perfectly smooth surfaces having the size and shape of the embossed front surfaces. Contacts 210, 310 and 410 have embossed surfaces with the same or similar ESA to SSA ratios and the contacts 110.

In at least some instances, including the illustrated embodiment, the rear surfaces 117 (FIG. 5) also have an ESA that is at least twice the SSA of the associated contacts 110. Put another way, the ESA to SSA ratio is greater than that of conventional conductive members and is at least 2.0 in the exemplary implementations. For example, the ESA to SSA ratio of the front surfaces 116 and rear surfaces 117 in other implementations may be at least 3.0, or at least 4.0, or at least 5.0, or at least 6.0, or at least 7.0, or at least 8.0, or at least 9.0, or at least 10.0.

One way to quantify the effective surface area of a surface is the average roughness (Ra) of that surface. The average roughness of the front surfaces 116 of contacts 110, 210, 310, 410 may be within a range of 100 nm to 800 nm in some implementations, may be within a range of 400 nm to 700 nm in other implementations, may be within a range of 500 nm to 600 nm in other implementations, and may be 566 nm in one specific implementation. For purposes of comparison, platinum-iridium foil may have a surface roughness of about 28 nm prior to a surface roughening/embossing process described below.

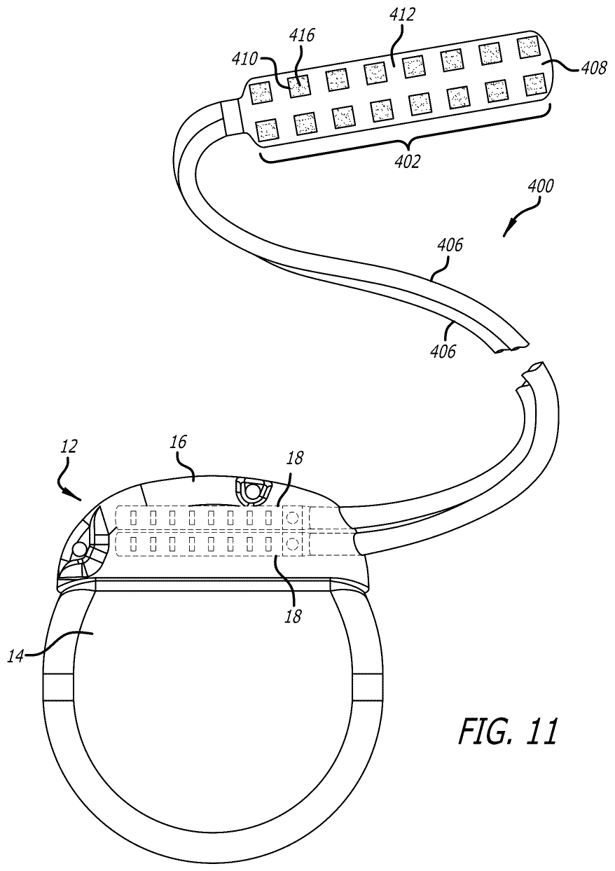

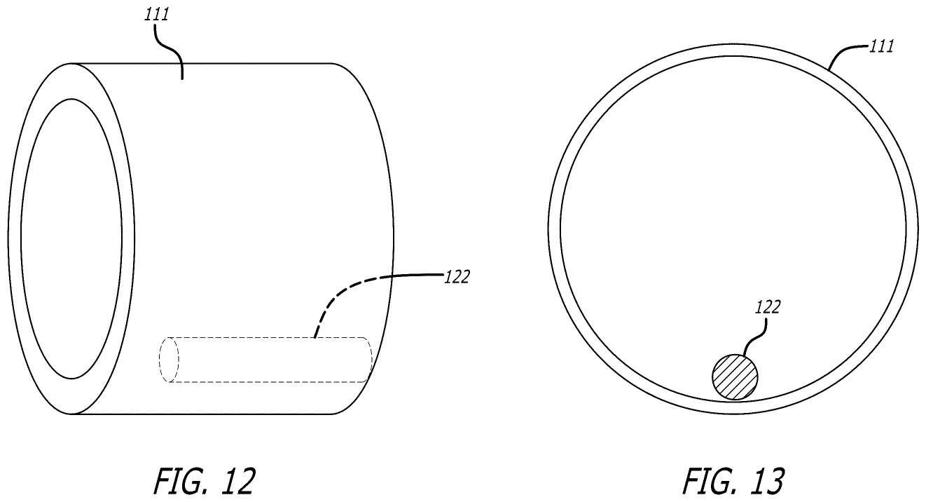

With reference to FIGS. 12-27, the contacts (and/or the conductive materials used to produce the contacts) are embossed to increase the surface roughness and the effective surface area of the conductive material from its pre-processed state to achieve the desired ESA to SSA ratios.

There are several advantages associated with an ESA to SSA ratio that is greater than that of conventional contacts and, for example, is at least 2.0. As compared to an otherwise identical electrode with conventional contacts, the stresses applied to the embossed contacts will be less likely to result in delamination or dislodgement from the substrate since the embossed surface on the underside of the contact permits better adhesion to the substrate than a non-embossed surface. Further, embossed contacts with higher ESA to SSA ratios can safely deliver an increased amount of stimulation energy (i.e., higher current density) through a window of a given size at a given or lower voltage, have reduced electrode-electrolyte impedance (thereby reducing power consumption by the stimulation circuitry and increasing IPG battery life), and have increased capacitance. It should also be noted that reducing electrode voltage reduces the likelihood of irreversible electrode reactions, such as electrolysis, which may lead to tissue irritation and/or damage. Increasing the effective surface area of the contacts also reduces edge effects, i.e., high current densities at the electrode edges, and the corrosion and/or tissue damage associated therewith.

As alluded to above, the contacts 110, 210, 310, or 410 may be formed from a tubular (or “ring-shaped”) workpiece 111 (FIG. 12) that is processed in such a manner that the effective surface area is increased. To that end, and referring to FIGS. 12-13 and FIGS. 20-21, the pre-processing tubular workpiece 111 can be formed from platinum-iridium and has a relatively smooth, visually shiny outer surface that will define the outer surfaces of the contacts formed from the workpiece post-processing. The tubular workpiece 111 can be pre-assembled with a wire 122 that will electrically couple the resulting contact 110 (or 210, 310, or 410) with the lead body 104 (or 204, 304, 404).

The following paragraphs provide a detailed description of a few ways in which the embossing process may be implemented, it being understood that recited specifics relating to the shape of the contacts, the tools used, and the structure of the final electrode may be changed without extending beyond the scope of the present invention.

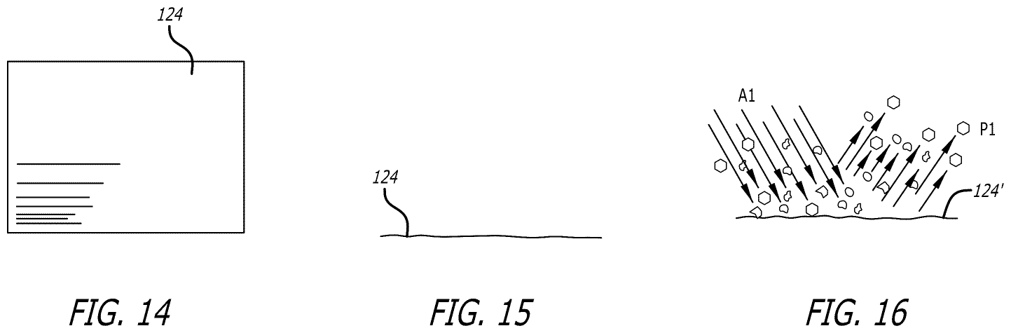

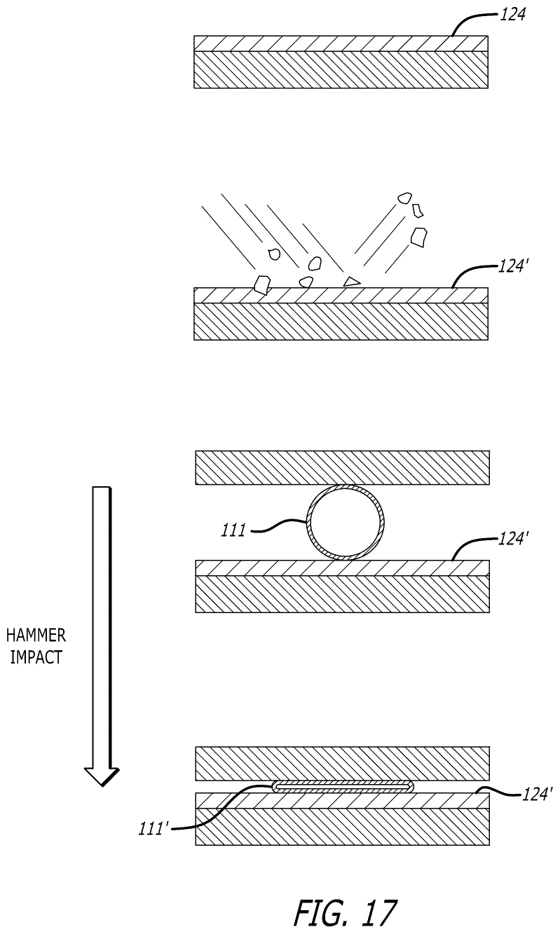

FIGS. 14-16 depict a representation of a tooling surface 124 undergoing a roughening process which occurs prior to the embossing process. It should be noted that any known method may be used to roughen tooling surface 124, and a depiction of grit blasting in FIGS. 14-16 is merely provided as one such example. Turning to FIG. 16, and as used herein, “grit blasting” is the shooting of a stream of small particulates P1 at a surface in a jet of air A1 and may be used to alter or clean surface 124. Grit blasting parameters such as air pressure, the material from which the particles are formed, size and size distribution, particle feed rate, particle shape, nozzle orifice diameter, air pressure, working distance and/or length of blasting time, may be selected and optimized to achieve the intended result. The grit blasting transforms the pre-processing tooling surface 124, where the outer surface of the tooling surface 124 has a relatively smooth, shiny state, to the post-processing tooling surface 124′ illustrated in FIGS. 16 and 17. The outer surface may be transformed to the finely textured, visually matte state in a single grit blasting process. The tubular workpiece 111 may then be pressed-upon or rolled-across the textured tooling surface 124′ to emboss at least one of the front contact surface 116 or rear contact surface 117 with the surface pattern.

The tooling surface 124 may be formed from any material but should be formed from a resilient metal material having a hardness that exceeds that of the workpiece. In one example, the tooling surface 124 is formed from a section of A-1 steel.

Advantageously, the textured tooling surface 124′ may be used many times to emboss multiple different workpieces, thereby reducing production cost and complexity relative to individually grit-blasting (or otherwise roughening) each piece of conductive material.

After embossing the tubular workpiece 111, embossed tubular workpiece 111′ may then be compressed or flattened into a substantially flat, rectangular embossed workpiece 111′, as shown in FIGS. 17 and 20-21. In other instances, processes other than grit blasting may be used to texturize the tooling surface, and the contacts 110, 210, 310, 410 may be similarly embossed via pressure and/or movement relative to the tooling surface and incorporated into electrodes with ESA to AP ratios that are at least 2.0 as described above.

FIGS. 17-23 depict additional embodiments for embossing methods in accordance with the present invention. With specific reference to FIG. 17, tubular workpiece 111 is placed upon the roughened tooling surface 124′ and sandwiched between another structure, which may or may not be roughened in a manner like tooling surface 124′. In this instance, a force, such as a hammer impact, is applied to the tooling structure to simultaneously emboss an outer surface of the tubular workpiece 111 while flattening it into a substantially rectangular embossed workpiece 111′, like that which is shown in FIG. 21.

Still referring to FIG. 17, magnified views of the tooling surface 124, 124′ are shown before and after a pre-fabrication grit blasting procedure to demonstrate the roughened topography achieved. Further, a magnified views of the surfaces of the tubular workpiece 111 and embossed tubular workpiece 111′ are respectively shown before and after embossing on the roughened tooling surface 124′. Following the embossing process, the effective surface area of the embossed tubular workpiece 111′ is increased, and such workpieces are combined with a substrate 108, 208, 308, 408 to form embossed contacts.

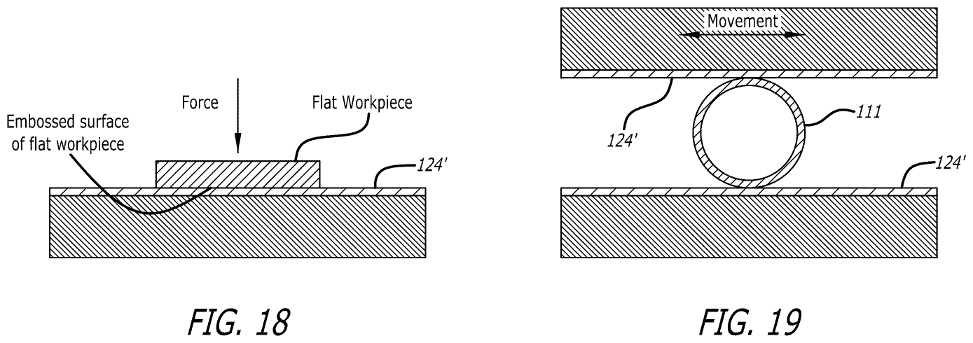

FIGS. 18-23 depict additional examples of methods for embossing electrically conductive workpieces prior to being assembled with an electrode, such as, e.g., a nerve cuff 102, helical electrode 202, linear electrode 302, or paddle electrode 402.

The embossing method of FIG. 18 is similar to that of FIG. 17, wherein a workpiece of electrically conductive material is placed upon a textured tooling surface 124′, and a force is applied vertically (i.e. orthogonal to the plane of tooling surface 124′) to emboss a surface of the electrically conductive workpiece with a surface pattern. In this instance, the electrically conductive workpiece is already substantially flat and does not comprise tubular workpiece 111.

Referring to FIG. 19, another example of a process for embossing a tubular workpiece 111 is shown wherein the tubular workpiece 111 of electrically conductive material is sandwiched between two roughened tooling surfaces 124′. In this instance, one or both tooling surfaces 124′ are configured to move laterally along a plane tangent to the outer surface of the tubular workpiece 111 such that the workpiece 111 is rolled therebetween to emboss an outer surface with the surface pattern. Advantageously, this method can allow for discrete portions of the outer surface, such as the front contact surface 116 or rear contact surface 117 (FIGS. 5 and 21) to be embossed. Alternatively, the entirety of the outer surface of the workpiece 111 may be embossed.

Turning to FIGS. 19-20, an embossing method similar to FIG. 17 is shown, wherein a tubular workpiece 111 is embossed and flattened to form the embossed workpiece 111′, which will subsequently be incorporated into an electrode to form a contact. The difference in FIG. 20, however, is the inclusion of a conductive wire 122 which is inserted within tubular workpiece 111. After embossing the tubular workpiece surface, the workpiece can then be compressed and flattened around the conductive wire 122 to mechanically connect the wire to the embossed tubular workpiece 111′ which can be used as an electrode contact 110 (or 210, 410), by incorporating into a cuff, helical, or paddle electrode, respectively.

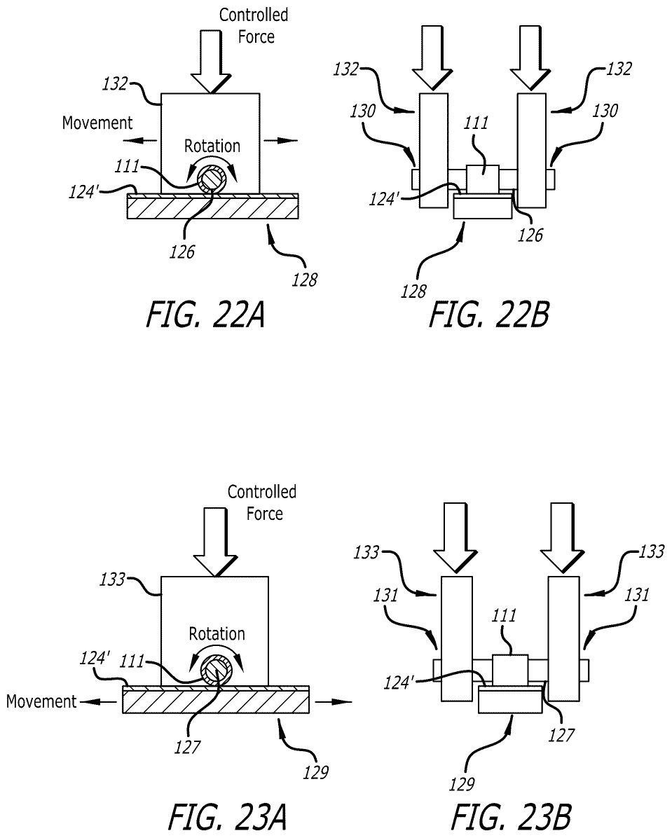

FIGS. 22A-22B and FIGS. 23A-23B depict additional embossing techniques in accordance with embodiments of the present disclosure wherein a tubular workpiece 111 is centrally supported on a pin 126 or 127 and rolled across a textured tooling surface 124′.

In FIGS. 22A and 22B, the textured tooling surface 124′ is secured upon a fixed, stationary plate 128. The tubular workpiece 111 is suspended above plate 128 by pin 126, which passes through an aperture of the workpiece 111, and respective ends of the pin 126 are secured within bearings 130 (FIG. 22B) of bearing supports 132. A controlled downward force upon bearing supports 132 presses the tubular workpiece 111 into contact with the textured tooling surface 124′, and lateral movements of the bearing supports 132 cause the tubular workpiece 111 to roll, thereby embossing an outer surface of the tubular workpiece 111 with the textured surface pattern. Similarly, the tubular workpiece 111 shown in FIGS. 23A and 23B is also supported by a pin 127 having distal ends disposed within bearings 131 of bearing supports 133.

However, while FIGS. 22A and 22B show a stationary plate 128 and a movable bearing support 132, FIGS. 23A and 23B show a movable plate 129 and a stationary bearing support 133. The ends of pin 127 are secured within bearings 131 (FIG. 23B) of bearing supports 133. In either embodiment, the tubular workpiece 111 may be embossed by the textured tooling surface 124′ on at least one discrete portion of the outer surface.

Although not shown in FIGS. 22A-23B, the embossed tubular workpiece 111′ may be optionally flattened after the embossing process to form a workpiece 111′ similar to that which is shown in FIG. 21.

Although an exemplary process of grit blasting was described in reference to pre-fabrication of a surface pattern on the tooling surface 124, any other known method for fabricating the surface pattern on the tooling surface 124 may be employed.

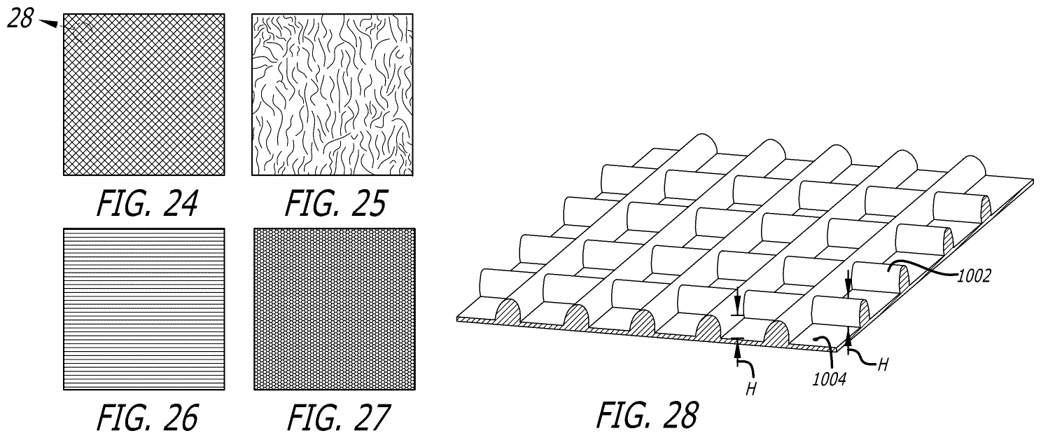

FIGS. 24-28 show multiple examples of surface topographies that could result from any one of the aforementioned embossing processes. The roughened topographies may comprise substantially irregular patterns (i.e. having micro-, macro- or nano-scale structures being irregularly sized, shaped or spaced). For example, an irregular roughened surface topography is shown in FIG. 25. Alternatively, the roughened surface topography may comprise regularly repeating geometric patterns like those which are shown in FIGS. 24, 26, 27, and 28. Referring to FIGS. 24 and 28 in particular, FIG. 24 is a top view of an exemplary regularly repeating surface pattern that could be embossed upon an electrode contact via an embossing process in accordance with the present disclosure, and FIG. 28 depicts a magnified isometric view of the surface pattern of FIG. 24. As shown in FIG. 28, the embossed surface pattern includes multiple protrusions 1002 which extend vertically outward to respective heights H above a reference plane 1004 on the outer surface of the embossed tubular workpiece 111′ (or contacts 110, 210, 310, 410).

The dimensions of the aforementioned electrodes, including the various elements thereof, may be any dimensions that result in the electrodes functioning as intended. With respect to the dimensions of substrate 108 of the exemplary nerve cuff electrode 102, and referring to FIG. 5, the cuff body is about 1.1 inches wide and about 0.34 inches long. As used herein in the context of dimensions, the word “about” means ±10-20%. The individual contacts 110 are depicted as being the same size in the illustrated implementation. In other implementations, the contacts 110, 210, 310, 410 may be different sizes.

Although the inventions disclosed herein have been described in terms of the preferred embodiments above, numerous modifications and/or additions to the above-described preferred embodiments would be readily apparent to one skilled in the art. It is intended that the scope of the present inventions extend to all such modifications and/or additions. The inventions include any and all combinations of the elements from the various embodiments disclosed in the specification. The scope of the present inventions is limited solely by the claims set forth below.

1. An electrode lead for transmitting electrical current to a nerve, the electrode lead comprising:
an elongate lead body having a proximal end and a distal end; and
an electrode including:
a biocompatible, pliable, electrically insulative substrate at the distal end of the lead body, and
at least one electrically conducive contact on the insulative substrate and including an embossed tissue contact surface.

2. The electrode lead claimed in claim 1, wherein
the insulative substrate comprises a nerve cuff with an inner surface configured to abut against the nerve; and
the at least one electrically conductive contact is associated with the inner surface in such a manner that the embossed tissue contact surface abuts against the nerve when the inner surface abuts the nerve.

3. The electrode lead claimed in claim 2, wherein
the nerve cuff is configured to be circumferentially disposed around the nerve and has a pre-set furled state that defines an inner lumen and is movable to an unfurled state.

4. The electrode lead claimed in claim 2, wherein
the nerve cuff is configured to be helically disposed around the nerve and has a pre-set furled state that defines an inner lumen and is movable to an unfurled state.

5. The electrode lead claimed in claim 1, wherein
the insulative substrate comprises a cylindrical substrate configured to be placed adjacent to the spinal cord; and
the at least one electrically conductive contact comprises a ring-shaped contact and the embossed tissue contact surface extends completely around the cylindrical substrate.

6. The electrode lead claimed in claim 1, wherein
the insulative substrate comprises a flat, rectangular substrate defining a front face; and
the at least one electrically conductive contact is associated with the front face in such a manner that the embossed tissue contact surface will abut against the nerve when the inner front face abuts the nerve.

7. The electrode lead claimed in claim 1, wherein
the at least one electrically conductive contact comprises a plurality of electrically contacts.

8. The electrode lead claimed in claim 1, wherein
the at least one electrically conductive contact has a smooth surface area defined by the outer perimeter of the embossed tissue contact surface; and
the embossed tissue contact surface defines an effective surface area that is bounded by the outer perimeter and that is at least twice the smooth surface area.

9. The electrode lead claimed in claim 1, wherein
the embossed tissue contact surface comprises a three-dimensional surface with a raised and/or depressed texture.

10. A method of forming an implantable neurostimulator electrode, the method comprising:
combining a plurality of electrically conductive members that include respective rear surfaces and embossed tissue contact surfaces with a biocompatible, pliable, electrically insulative substrate that includes a plurality of windows in such a manner that exposed portions of the embossed tissue contact surfaces are within the windows.

11. The method claimed in claim 10, wherein
the substrate comprises a nerve cuff with an inner surface configured to abut against the nerve; and
the windows are associated with the inner surface.

12. The method claimed in claim 11, wherein
the nerve cuff is configured to be circumferentially disposed around a nerve and has a pre-set furled state that defines an inner lumen and is movable to an unfurled state.

13. The method claimed in claim 11, wherein
the nerve cuff is configured to be helically disposed around a nerve and has a pre-set furled state that defines an inner lumen and is movable to an unfurled state.

14. A method of forming an electrode for an implantable neurostimulator, the method comprising:
placing electrically conductive material in physical contact with a tooling surface having a surface pattern;
embossing the surface pattern onto the electrically conductive material to form an electrically conductive contact with an embossed tissue contact surface; and
combining the electrically conductive contact with a biocompatible, pliable, electrically insulative substrate.

15. The method of claim 14, wherein
the electrically insulative substrate includes a window; and
the electrically conductive contact is combined with the electrically insulative substrate in such a manner that at least a portion of the embossed tissue contact surface is within the window.

16. The method of claim 14, wherein
the electrically insulative substrate comprises a cylindrical substrate configured to be placed adjacent to the spinal cord;
the electrically conductive material is ring-shaped;
the electrically conductive contact is ring-shaped; and
the ring-shaped electrically conductive contact is combined with the cylindrical substrate in such a manner that the embossed tissue contact surface extends completely around the cylindrical substrate.

17. The method of claim 14, wherein
the electrically conductive material comprises a substantially cylindrical ring;
embossing the surface pattern onto the electrically conductive material comprises causing the substantially cylindrical ring to roll upon the tooling surface, and
the method further comprises flattening embossed substantially cylindrical ring such that the embossed tissue contact surfaces are substantially rectangular prior to combining the electrically conductive contact with the electrically insulative substrate.
